(19) United States
(12) Patent Application Publication
(10) Pub. No.: US 2007/0191946 A1
(43) Pub. Date: Aug. 16, 2007

(54) Intervertebral spinal implant devices and methods of use

(75) Inventors: Eric Steven Heinz, Memphis, TN (US); Philippe E. Pare, Cordova, TN (US); Roy Lim, Germantown, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/343,954

(22) Filed: Jan. 31, 2006

(51) Int. Cl. A61F 2/44

(52) U.S. Cl. 623/17.11

(57) ABSTRACT

A spinal implant device used for the surgical treatment of a spinal disorder. The implant device may be a static device or a dynamic device. In one embodiment, the implant device is constructed of a radiolucent material with attached radiopaque markers. The markers may be constructed of the same radiolucent material and a radiopaque additive. In one embodiment, the implant device is constructed of a carbon nanostructure reinforced polymer. In one embodiment, the implant device has a porous bone interface surface. The pore density of the bone interface surface may vary up to a larger value in areas where the bone interface surface contacts a cortical bone portion of a vertebra.

### BACKGROUND

Intervertebral spinal implants are often used in the surgical treatment of spinal disorders such as degenerative disc disease, disc herniations, scoliosis and other curvature abnormalities, and fractures. Many different types of treatments are used. In some cases, spinal fusion is indicated to inhibit relative motion between vertebral bodies. In other cases, dynamic implants are used to preserve motion between vertebral bodies. Further, various types of implants may be used, including intervertebral and interspinous implants. Other implants are attached to the exterior of a vertebrae, whether it be at a posterior, anterior, or lateral surface of the vertebrae.

Some spinal implants use metal alloys including titanium, cobalt, and stainless steel. Unfortunately, metals such as these may tend to interfere or obscure MRI and X-ray images. Accordingly, non-metallic implant designs have become more popular. For example, implantable grade polyetheretherketone (PEEK) and other similar materials (e.g., PAEK, PEKK, and PEK) offer alternative solutions for implant device materials. However, even these materials have certain drawbacks. First, these base materials may not have the strength to survive long-term use, particularly in the spine where the implants may be subjected to substantial compressive loads. Second, these base materials, in their stock form, may not readily adhere to vertebral members, which may be important for long-term stability. Thirdly, these materials are generally radiolucent and not visible in X-ray imaging. X-ray imaging may be desirable during installation of the device and post-operation to check the condition of the implant. Accordingly, while implantable grade PEEK and other members of the PEK family may be an attractive material choice, various limitations of the base material may call for improvements to a spinal implant device that is made of these materials.

### SUMMARY

Illustrative embodiments disclosed herein are directed to a spinal implant device used for the surgical treatment of a spinal disorder. The implant device may be a static device or a dynamic device. In one embodiment, the implant device is constructed of a radiolucent material with attached radiopaque markers. The markers may be constructed of the same radiolucent material and a radiopaque additive. Different levels of radiopaque additive or different radiopaque additives may be used to construct the markers. The markers may be attached within, partially within, or exterior to the device. In one embodiment, the implant device is constructed of a carbon nanostructure reinforced polymer. The carbon nanostructures may be nanofibers, nanotubes, or nanospheres. In one embodiment, the implant device has a porous bone interface surface. The pore density of the bone interface surface may vary up to a larger value in areas where the bone interface surface contacts a cortical bone portion of a vertebra.

### BRIEF DESCRIPTION OF THE DRAWINGS

FIG. 1 is a side schematic view showing a portion of a spine and a spinal arthroplasty device according to one embodiment;

FIG. 2A is a posterior facing section view of a spinal arthroplasty device according to one embodiment;

FIG. 2B is a posterior facing section view of an exploded spinal arthroplasty device according to one embodiment;

FIG. 3 is an anterior/posterior view of an end plate of a spinal arthroplasty device according to one embodiment;

FIGS. 4A and 4B are lateral views of a nucleus of a spinal arthroplasty device according to one embodiment;

FIG. 5 is an anterior view of a nucleus of a spinal arthroplasty device according to one embodiment;

FIG. 6 is a superior view of a vertebra and various embodiments of an intervertebral implant; and

FIGS. 7A and 7B are posterior views of an intervertebral implant comprising a plurality of markers according to one embodiment.

### DETAILED DESCRIPTION

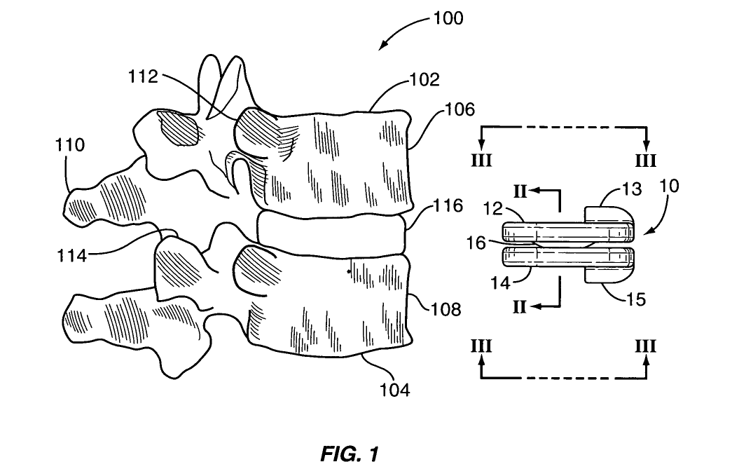

The various embodiments disclosed herein relate to a spinal implant device that may be used for the surgical treatment of a spinal disorder. FIG. 1 shows a lateral view of an exemplary spinal arthroplasty device 10 adjacent to a portion of a spine 100. Specifically, FIG. 1 shows two vertebrae 102, 104 and a disc 116 therebetween. Each vertebra 102, 104 includes a generally cylindrical body 106, 108 that contributes to the primary weight bearing portion of the spine 100. Further, each vertebra 102, 104 includes various bony processes 110, 112 extending posterior to the body 106, 108. Adjacent vertebrae 102, 104 may move relative to each other via facet joints 114 and due to the flexibility of the disc 116.

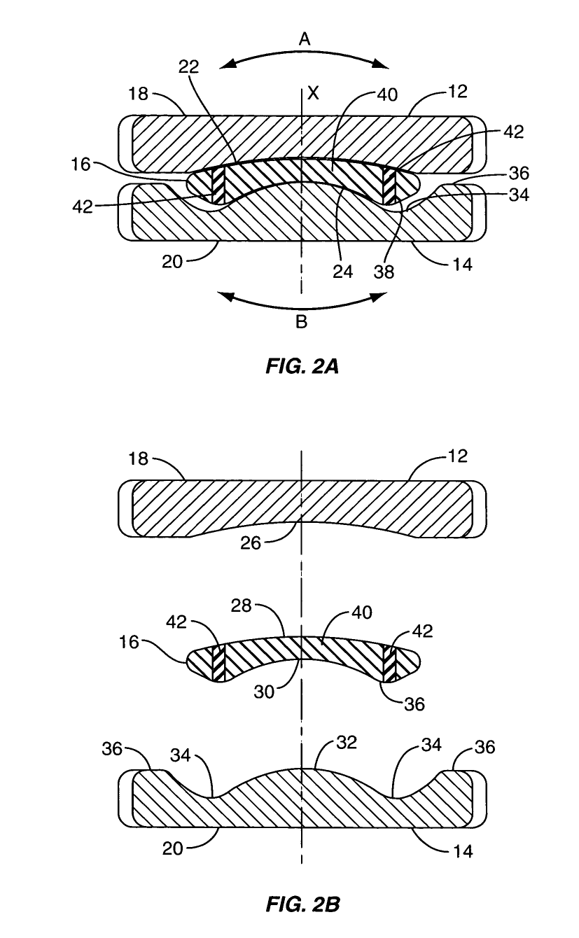

For instances where the disc 116 is herniated or degenerative, the entire disc 116 may be replaced with the spinal arthroplasty device 10. The spinal arthroplasty device 10 shown in FIG. 1 comprises three main components: a first end plate 12, a second end plate 14, and a nucleus 16. The cross section of the spinal arthroplasty device 10 provided in FIGS. 2A and 2B shows the configuration of the three components 12, 14, 16. FIG. 2A represents the spinal arthroplasty device 10 in an assembled configuration while FIG. 2B provides an exploded view of the components taken along the same section line II-II from FIG. 1. In the orientation shown, the first end plate 12 is a superior end plate while the second end plate 14 is an inferior end plate. However, it should be understood that the orientations may be reversed if so desired.

Each end plate 12, 14 may include a respective bone interface surface 18, 20 that is placed in contact with a corresponding body 106, 108 of a vertebral member 102, 104. In addition, each end plate 12, 14 may include a respective anchor 13, 15 that fits within a corresponding recess (not shown) in the vertebrae 102, 104. The vertebrae 102, 104 may require some amount of surgical preparation to accept the end plates 12, 14. This may include contouring to match the bone interface surfaces 18, 20 and/or bone removal to create recesses into which the anchors 13, 15 are inserted.

The nucleus 16 is positioned between the end plates 12, 14. The interface 22 between the nucleus 16 and the first end plate 12 is a sliding interface that allows for sliding motion of the nucleus 16 relative to the first end plate 12. This sliding motion is illustrated by the arrow labeled A in FIG. 2A. This arrow A suggests motion in a direction parallel to the page. However, the interface 22 between the nucleus 16 and first end plate 12 is substantially spherical. Specifically, the interface 22 is defined in part by the mating surfaces 26, 28 (see FIG. 2B) on the first end plate 12 and the nucleus 16, respectively. The first end plate bearing surface 26 and the first nucleus bearing surface 28 are spherical surfaces. Further, since sliding motion is contemplated at the interface 22 between these surfaces 26, 28, each may be polished to a fine surface finish. In one embodiment, the spherical radii of the first end plate bearing surface 26 and the first nucleus bearing surface 28 are the same or substantially similar. Consequently, the sliding motion at the interface 22 may occur in virtually all directions relative to a central axis X. In an alternative embodiment, the mating surfaces 26, 28 may be cylindrical, thus limiting sliding motion to the direction of the arrow labeled A.

A similar interface surface 24 (FIG. 2A) exists between the nucleus 16 and the second end plate 14. The interface 24 is defined in part by the mating surfaces 30, 32 (identified in FIG. 2B) on the nucleus 16 and the second end plate 14, respectively. In the example shown, the second nucleus bearing surface 30 and second end plate bearing surface 32 are also spherical surfaces. Consequently, the sliding motion at the interface 24 (identified by arrow B) may occur in virtually all directions relative to a central axis X.

The spherical radii of the second nucleus bearing surface 30 and the second end plate bearing surface 32 may be the same or substantially similar to each other. However, the spherical radius of surfaces 30, 32 may be generally smaller than the spherical radius of surfaces 26, 28. For example, in one embodiment, the spherical radius of surfaces 30, 32 may be about 20-25 mm while the spherical radius of surfaces 26, 28 may be about 70-75 mm. Further, since sliding motion is contemplated at the interface 24 between surfaces 30, 32, each may be polished to a fine surface finish.

The second end plate 14 differs slightly from end plate 12 in that the second end plate 14 includes an annular recess 34 between the second end plate bearing surface 32 and an outer annular rim 36. The size and location of the annular recess 34 corresponds with the shape at the perimeter of the nucleus 16. The nucleus 16 includes a generally disc-shaped configuration with the outer perimeter 38 having a thickness that is larger than the innermost portion 40 adjacent to the central axis X (between bearing surfaces 28, 30). As the bearing surfaces 30, 32 slide over one another, the enlarged outer perimeter 38 of the nucleus approaches and enters the annular recess 34. However, the range of sliding motion is limited by the outer annular rim 36, which inhibits further sliding motion between the nucleus 16 and the second end plate 14. Thus, the nucleus 16 may remain in a sandwiched configuration between the first and second end plates 12, 14.

FIGS. 2A and 2B also show a plurality of markers 42 disposed within the nucleus 16. In one embodiment, the nucleus 16 is comprised of an implantable grade PEEK material. One example of a suitable medical grade material is marketed as PEEK®-Optima available from Invibio, Inc. in Greenville, S.C., USA. Suitable alternative materials for the nucleus 16 may comprise other radiolucent polymer materials, including but not limited to polyether ketone (PEK), polyether ketone ketone (PEKK), and polaryl ether ketones (PAEK). Each of these alternatives may be radiolucent, which generally refers to that characteristic which prevents the material from appearing in plain film radiographic images when implanted within a patient. Therefore, one or more radiopaque markers 42 may be incorporated into the nucleus 16 to make the nucleus 16 visible in X-ray images.

It is generally understood that biocompatible metals, including stainless steel, titanium, gold, and platinum may be used to create marking pins, wires, and spheres as X-ray markers so that the position of the implant can be identified in a plain film radiograph. However, in the present embodiment, the radiopaque markers 42 are comprised of PEEK (or PEK, PEKK, PAEK) that is impregnated with a radiopaque additive such as barium sulfate or bismuth compounds. In one embodiment, the markers 42 are comprised of PEEK having a 4-30% by weight mixture of barium sulfate. This may be done for several reasons. First, the addition of a radiopaque substance means the markers 42 will be visible in X-ray images. This is due to the fact that the markers 42 are characterized by a radiolucency that is greater than that of the nucleus 16. Second, the barium sulfate is MRI compatible unlike many metallic markers that can create MRI and CT distortions. Third, the substrate material for the markers 42 is substantially the same as the rest of the nucleus, which minimizes the effects of corrosion that is produced at the interface between dissimilar materials. That is, the interface between the markers 42 and nucleus may be less prone to corrosion since the substrate materials are the same.

The markers 42 are shown in FIGS. 2A, 2B oriented parallel to the central axis X. This orientation may provide optimal visibility in lateral, anterior, and posterior films. Furthermore, orienting the markers 42 parallel to one another may provide some indication that the nucleus 16 is damaged in the event a radiograph shows the markers 42 in some orientation other than parallel to one another. However, this does not preclude the use of markers 42 oriented in other directions. Also, the markers 42 are disposed in the enlarged outer perimeter 38 of the nucleus, thus allowing for a longer marker 42. The markers 42 may be incorporated into the nucleus 16 using a variety of techniques. For instance, the markers 42 may be bonded in place, molded into the nucleus 16, or press fit into machined apertures (not explicitly shown) in the nucleus 16.

FIG. 3 shows a view of the first or second end plate 12, 14 according to the view lines III-III shown in FIG. 1. Two sets of view lines III-III are provided in FIG. 1. Thus, the view shown in FIG. 3 depicts either a top view of the first end plate 12 or a bottom view of the second end plate 14. For purposes of this disclosure, the bone interface surfaces 18, 20 and the corresponding anchors 13, 15 may be considered substantially similar. In actuality, the bone interface surfaces 18, 20 and anchors 13, 15 may be different to accommodate the anatomy of the vertebrae 102, 104. However, the one view shown in FIG. 3 will suffice for the following discussion.

Similar to the nucleus 16, FIG. 3 shows a plurality of markers 42a, 42b disposed within the end plate 12, 14. In one embodiment, the end plate 12, 14 is comprised of an implantable grade PEEK material. Suitable alternatives for the end plate 12, 14 may comprise other radiolucent polymer materials selected from the polyether ketone (PEK) family, including but not limited to polyether ketone ketone (PEKK) and polaryl ether ketones (PAEK). Each of these alternatives may be radiolucent. Therefore, the radiopaque markers 42a, 42b may be incorporated into the end plate 12, 14 to improve the visibility of the end plate 12, 14 in X-ray images.

In addition, the first radiopaque markers 42a may be comprised of a radiolucent polymer and a first concentration of barium sulfate. As a non-limiting example, the first concentration may be about 4% by weight. The second radiopaque markers 42b may be comprised of a radiolucent polymer and a second radiopaque material, such as a bismuth compound. Alternatively, the second radiopaque markers 42b may be comprised of a radiolucent polymer and a second concentration of barium sulfate. As a non-limiting example, the second concentration may be about 6% by weight. The different compositions for the first markers 42a and the second markers 42b may allow one to distinguish between the first markers 42a and second markers 42b in a radiograph.

As with the nucleus 16, the markers 42 may be positioned in thicker regions of the end plate 12, 14 and extend between a top and bottom side of the nucleus. Thus, for the first end plate 12, the markers 42 may be positioned outside of the first end plate bearing surface 26 (see FIG. 2B). In the case of the second end plate 14, the markers may be positioned outside of the annular recess 34 in the vicinity of the outer annular rim 36 (also see FIGS. 2A, 2B). In addition, it may be desirable to include a marker within the anchor 13, 15. As above, the markers 42 may be oriented parallel to one another to provide some indication that the first or second end plates 12, 14 are damaged in the event a radiograph shows the markers 42 in some orientation other than parallel to one another.

FIG. 3 also shows a pair of dashed lines 44, 46 that generally divide the bone interface surface 18, 20 into a plurality of regions 48, 50, 52. The bone interface surface 18, 20 is a generally porous surface. As used herein, the terms pore and porosity are used to represent minute openings, especially about the exterior of the implant surface through which bony matter may grow. The pores may be formed as projections or recesses and may be interconnected or separate from one another. The pores may be formed using a post-processing technique such as blasting, etching, and coating, such as with hydroxyapatite. The bone interface surface 18, 20 may also include growth-promoting additives such as bone morphogenetic proteins. Alternatively, the pores may be incorporated into a molding process. The pore density is advantageously ideal to promote bone integration to the respective vertebrae 102, 104. Further, the pore density is generally different in the different regions 48, 50, 52. For instance, a larger pore density (i.e., higher porosity) exists about the periphery of the end plate 12, 14 at the outermost region 48. The greater porosity about the periphery of the end plate 12, 14 may permit bone growth in the regions of the body 106, 108 of vertebrae 102, 104 that are characterized by denser cortical bone.

By comparison, the pore density in regions 50 and 52 are incrementally smaller than in the outermost region 48. These intermediate 50 and innermost 52 regions correspond to areas with a thin bone plate and increasingly cancellous bone portions of the vertebral bodies 106, 108. The varying porosity of the bone interface surfaces 18, 20 may also be incorporated as a gradient that is not marked by definite transitions such as dashed lines 44, 46. Instead, the porosity may vary gradually in a direction away from the outer perimeter of the bone interface surfaces 18, 20.

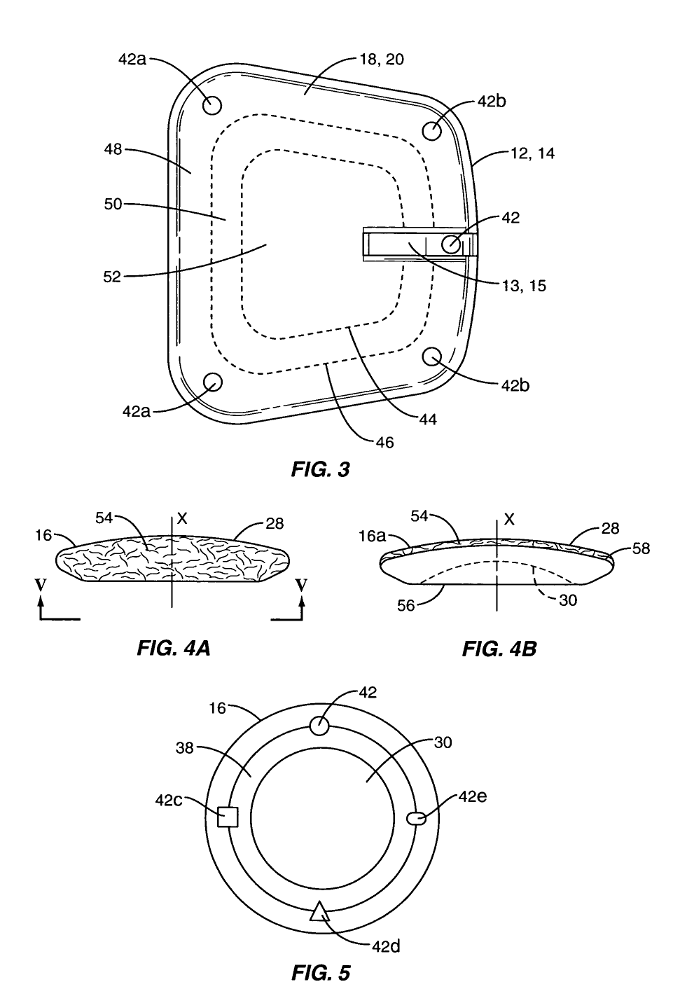

FIG. 4A shows a lateral view of an exemplary nucleus 16 for use in the spinal arthroplasty device 10. As discussed before, the nucleus 16 may be comprised of an implantable grade PEEK material or other radiolucent polymer materials selected from the polyether ketone (PEK) family, including but not limited to polyether ketone ketone (PEKK) and polaryl ether ketones (PAEK). The compressive strength and wear resistance of the nucleus 16 may be improved with a formulation that includes a variety of additives. In one embodiment, the additive includes carbon fibers, which are graphically illustrated in FIG. 4 as elongated strands 54. The elongated depiction of the fibers 54 is provided merely as a graphical representation of the fibers 54. In actuality the length of the strands may be small, such as in the range between about 50-100 microns. In one embodiment, the fibers are carbon nanostructures such as nanofibers or nanotubes. In another embodiment, the carbon additives are nanospheres of carbon, including Buckminsterfullerenes, which are often referred to colloquially as Buckyballs. In one or more embodiments, the substrate material may be filled with between about 2-15% by weight carbon nanofibers. The nanostructures may be formed by a process that involves growth from a metal catalyst particle. Also, the carbon nanostructures may be vapor grown hollow nanofibers. In one embodiment, the fibers 54 may have a mean diameter between about 125 and 185 nm. In one embodiment, the nucleus 16 is comprised of a 10% weight vapor grown carbon nanofiber PEEK composite. One example of a suitable carbon nanofiber is Pyrograph III, supplied by Applied Sciences, Inc. of Cedarville, Ohio, USA.

Implantable grade PEEK generally includes a strong bond with carbon nanofibers 54. Thus, fiber and substrate wear particles may be reduced. Generally, the carbon nanofibers may act as a lubricant, and in contrast to conventional carbon fiber fillers, may not produce a roughening effect at the surface of the nucleus 16. With longer fibers, the orientation of the fibers may affect wear resistance. However, the comparatively small size of carbon nanofibers or nano-spheres may contribute to an improvement in wear characteristics that may be independent of orientation. However, the orientation of the carbon nanofibers may be controlled to provide varying material characteristics. The overall improvements may be apparent, not only in the nucleus 16, but also in the mating bearing surfaces 26, 32 on the first and second end plates 12, 14, respectively.

The nucleus 16 may be constructed from an injection molding process whereby the carbon nanofibers 54 are homogeneously incorporated and dispersed in the substrate. Alternatively, the material may be formed through an extrusion process. Various process variables in an injection molding process may be altered to control the surface characteristics of the nucleus 16. As those skilled in the art of composite manufacturing will understand, temperature, pressure, flow rates, and cooling times may be adjusted to adjust the composition of the outermost layer of the nucleus 16. Through proper control, the bearing surfaces 28, 30 of the nucleus may be produced resin rich. That is, fewer additives such as the carbon nanofibers 54 may be disposed at or near the bearing surfaces 28, 30. In on embodiment, the bearing surfaces 28, 30 are resin rich to a depth of less than about 0.025 inch.

In one embodiment, the bearing surfaces 28, 30 are constructed to tightly controlled tolerances. For instance, the bearing surfaces 28, 30 may have a surface finish that is about 2 micrometers or less. Also, the bearing surfaces 28, 30 may be constructed as substantially spherical surfaces. In this case, the bearing surfaces 28, 30 may have a sphericity that is about 20 micrometers or less. In one embodiment, the sphericity may be measured over the entire bearing surface 28, 30. In an alternative embodiment, the sphericity may be measured over some solid angle that is less than the entire bearing surface 28, 30. The bearing surfaces 28, 30 may be produced through a machining, polishing, or molding process.

FIG. 4B illustrates an alternative configuration for the nucleus 16a that is comprised of a body portion 56 that is covered by an outer layer 58. The body portion 56 may be comprised of PEEK material while the outer layer 58 comprises a carbon nanofiber reinforced PEEK material as described above. A carbon-fiber reinforced outer layer 58 may cover bearing surface 30 as well, although this outer layer 58 is not visible in FIG. 4B.

FIG. 5 illustrates a bottom view of the nucleus 16 according to the view lines V-V shown in FIG. 4. This particular view illustrates the second nucleus bearing surface 30 and the enlarged outer perimeter 38. FIG. 5 also shows a plurality of markers 42, 42c, 42d, 42e incorporated into the enlarged outer perimeter 38 of the nucleus 16. In FIGS. 2A, 2B, the markers 42 were illustrated as elongated members. As FIG. 5 shows, the markers 42, 42c, 42d, 42e may be provided with a different cross section. Thus, the cross section may be circular (marker 42), rectilinear (marker 42c), triangular (marker 42d), oval (marker 42e) or other shapes. Non-circular cross sections may prevent unwanted rotation of the marker 42 within the body of the nucleus 16. In other embodiments, the markers 42 may be spherical or block shaped as opposed to the elongated markers 42 heretofore described. A more compact marker 42 may advantageously allow the markers 42 to be inserted into thinner sections of the nucleus 16 or end plates 12, 14.

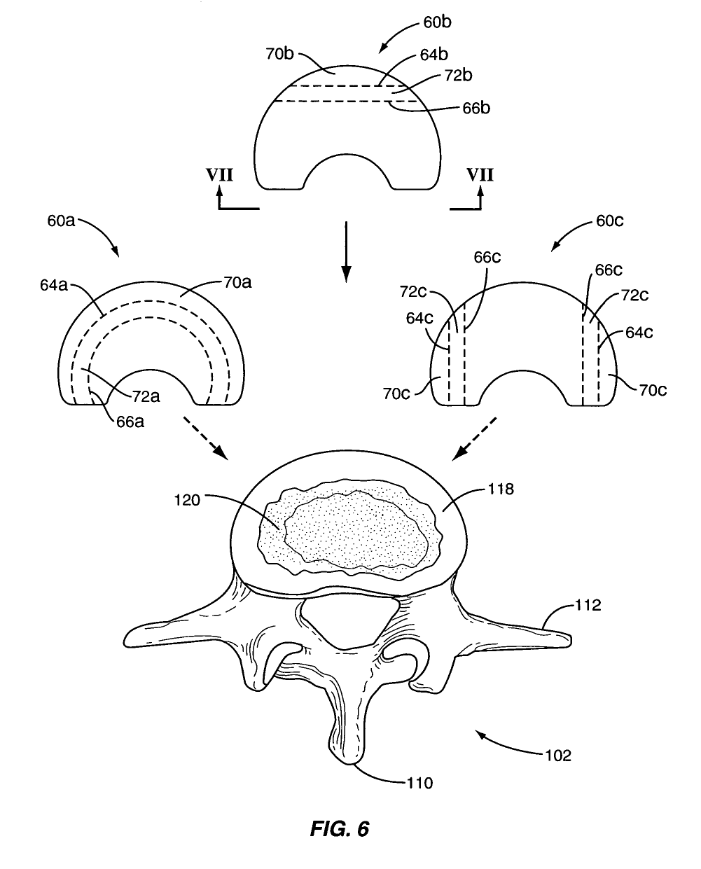

FIG. 6 shows a superior (or inferior) view of a vertebra 102 and various embodiments of an intervertebral implant 60a-c. The intervertebral implants 60a-c are depicted as a prosthetic disc that is a relatively static device as compared to the dynamic arthroplasty device 10 heretofore described. The implants 60a-c shown may be inserted between vertebral bodies 102, 104 to allow limited motion or as a spacer during spinal fusion procedures. For either case, the implants 60a-c may be provided with a generally porous surface to promote bone ingrowth. Specifically, each embodiment of the vertebral implant 60a-c includes a varying porosity that is illustrated by the dashed lines 64a-c, 66a-c. For each embodiment 60a-c, the outer dashed line 64a-c defines a first region 70a-c of a face of the implant 60a-c that supports a cortical rim 118 of the vertebra 102. This first region 70a-c is characterized by a relatively large pore density to permit greater bone ingrowth, especially in the vicinity of the cortical rim 118. The various embodiments of the first region 70a-c span varying amounts of the overall perimeter of the implant 60a-c. For instance, first region 70a spans substantially all of the perimeter of implant 60a that is in contact with the cortical rim 118 of vertebra 102. By comparison, first regions 70b-c span some lesser amount of the perimeter of implants 60b-c. The inner dashed line 66a-c defines at least a second region 72a-c that is characterized by a pore density that is less than that of the first region 70a-c. The lower pore density in this inner second region 72a-c may be appropriate due to a lower capacity for ingrowth in the areas where thin vertebral end plates 120 cover cancellous bone in vertebra 102.

As discussed above, the regions of varying pore densities may be formed using a post-processing technique such as blasting, etching, and coating, such as with hydroxyapatite. The bone interface surface 18, 20 may also include growth-promoting additives such as bone morphogenetic proteins. Furthermore, in the embodiments illustrated in FIGS. 3 and 6, the regions of varying pore density may be formed through the use of carbon nanofibers. Carbon nanofibers at the various bone interface surfaces may provide increased, select, osteoblast adhesion on carbon nanofiber compositions. Thus, regions 48, 50, 52 in FIG. 3 and regions 70a-c and 72, a-c from FIG. 6 may be formed through compression molding carbon nanofibers onto the endplates 12, 14 and implants 60a-c. In an alternative embodiment, the carbon nanofibers may be applied through a plasma spray process.

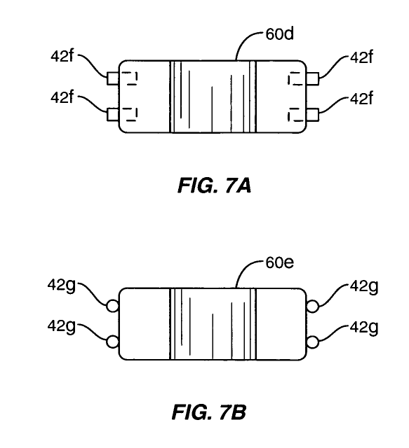

FIGS. 7A and 7B depict posterior views of an intervertebral implant 60d-e according to the view lines VII-VII shown in FIG. 6. As disclosed above, the implants 60d-e may be constructed of PEEK or a suitable derivative while the markers 42f-g are constructed of PEEK and a radiopaque additive. In the embodiment shown in FIG. 7A, the markers 42f are attached to the implant 60d by partially inserting the marker 42f into the implant 60d. By comparison, FIG. 7B shows a plurality of radiopaque markers 42g that are attached to the implant 60e through bonding, heating, ultrasonic welding or other process capable of attaching the markers 42g to the implant 60e.

The various Figures and embodiments disclosed herein have depicted an intervertebral device that is inserted between vertebral bodies. However, the teachings disclosed are certainly applicable to other types of spinal implant devices, including interspinous spacers, rods, plates, and other devices that are attached about the exterior of a vertebrae 102, 104.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. For example, embodiments described above have contemplated a nucleus 16 that includes first and second bearing surfaces 28, 30 that are curved in the same direction. In other embodiments, the first and second bearing surfaces 28, 30 of the nucleus may be oppositely curved. Further, as suggested above, the first and second end plates may be inverted as appropriate. That is, the spherical interface surfaces 22, 24 may curve upwards if desired. The present embodiments are, therefore, to be considered in all respects as illustrative and not restrictive, and all changes coming within the meaning and equivalency range of the appended claims are intended to be embraced therein.

What is claimed is:

1. An implant device comprising:
a body constructed from a first radiolucent material; and
a first marker positioned within the body, the first marker constructed from the radiolucent material and having a radiopaque additive.

2. The implant device of claim 1 further comprising first and second end plates on each side of the body.

3. The implant device of claim 1 further comprising a second marker oriented substantially parallel to the first marker.

4. The implant device of claim 1 further comprising a second marker constructed from a different concentration of radiopaque additive as compared to the first marker.

5. The implant device of claim 1 further comprising a second marker constructed from a different radiopaque additive as compared to the first marker.

6. The implant device of claim 1 wherein the marker extends from a first side of the body to a second side of the body.

7. The implant device of claim 1 wherein the first material is completely radiolucent when viewed within a patient through an x-ray device.

8. The implant device of claim 1 wherein the body comprises a protrusion to engage a bony surface, the first marker positioned within the protrusion.

9. An implant device comprising:
a body constructed from a first material having a first radiolucency; and
a first marker positioned within the body, the first marker constructed from the first material and a second material, the second material having a second radiolucency greater than the first material.

10. The implant device of claim 9 further comprising a second marker constructed from the first material and a third material having a third radiolucency different than the second material.

11. The implant device of claim 9 wherein barium sulfate is the second material.

12. The implant device of claim 11 wherein between about 4-6 percent by weight barium sulfate is added to the first material.

13. The implant device of claim 9 further comprising a second marker constructed from the first material and a different amount of the second material as compared to the first marker.

14. A method of making an implant comprising the steps of:
forming a body from a radiolucent material;
forming a marker from at least the radiolucent material and a radiopaque material; and
attaching the marker to the body.

15. The method of claim 14 wherein attaching the marker to the body comprises molding the body around the marker.

16. The method of claim 14 wherein attaching the marker to the body comprises pressing the marker into the body.

17. The method of claim 14 wherein attaching the marker to the body comprises adhering the marker to the body.

18. The method of claim 14 wherein attaching the marker to the body comprises embedding the marker within the body.

19. The method of claim 14 further comprising forming a second marker from the radiolucent material and a different radiopaque material and attaching the second marker to the body.

20. The method of claim 14 further comprising forming a second marker from the radiolucent material and a different quantity of the radiopaque material and attaching the second marker to the body.

21. The method of claim 14 further comprising forming a protrusion extending from the body to engage a bony surface and inserting the marker into the protrusion.

22. An implant device comprising:
an outer surface having a first region and a second region, each region having a common construction, the first region having a pore density that is greater than the second region.

23. The implant device of claim 22 wherein the first region is disposed about the perimeter of the device.

24. The implant device of claim 23 wherein the first region spans substantially a full perimeter of the implant device.

25. The implant device of claim 23 wherein the first region spans a portion of a perimeter of the implant device.

26. The implant device of claim 22 wherein the second region is Inside of first region.

27. The implant device of claim 22 wherein the common construction comprises carbon nanofibers.

28. An implant device comprising:
a body sized to be inserted within an intervertebral space between a first and second vertebra, the body having a face to contact one of the vertebra, the face having a first region that substantially aligns with a cortical rim of the vertebra and a second region inward from the first region, the first region having a pore density that is greater than the second region.

29. The implant device of claim 28 wherein the first region spans substantially all of the cortical rim.

30. The implant device of claim 28 wherein the first region spans a portion of the cortical rim.

31. The implant device of claim 28 wherein the first region is disposed at a perimeter of the implant device.

32. The implant device of claim 28 wherein the second region is disposed away from a perimeter of the implant device.

33. A method of making an implant comprising:
forming an outer layer on a support surface of the implant;
creating a first region on the outer layer having a first pore density; and
creating a second region on the outer layer having a second pore density that is different than the first density.

34. The method of claim 33 further comprising merging the first region into the second region.

35. The method of claim 33 wherein the second region is disposed inward of the first region.

36. The method of claim 33 further comprising forming a gradient transition between the first region and the second region.

37. The method of claim 33 wherein the step of forming an outer layer on a support surface of the implant further comprises applying carbon nanofibers to the outer layer.

38. A implant that provides for dynamic motion in the spine comprising:
a body having a bearing surface to allow relative vertebral motion, the body constructed of a polymeric matrix having carbon nanostructures.

39. The implant of claim 38 wherein the bearing surface is a polished surface.

40. The implant of claim 38 wherein the bearing surface has a surface roughness of less than about 2 micrometers.

41. The implant of claim 38 wherein the bearing surface has a sphericity of less than about 20 micrometers.

42. The implant of claim 38 wherein the carbon nanostructures have a diameter of less than about 185 nm.

43. The implant of claim 38 wherein the carbon nanostructures are nanotubes.

44. The implant of claim 38 wherein the carbon nanostructures are nanospheres.

45. The implant of claim 38 wherein the carbon nanostructures are nanofibers.

46. The implant of claim 38 wherein the body is further constructed of PEEK.

47. A implant that provides for dynamic motion in the spine comprising:
an end plate having a bone interface surface and a first bearing surface; and
a nucleus having a second bearing surface that slidingly engages the first bearing surface to allow relative vertebral motion, the nucleus constructed of a polymeric matrix having carbon nanostructures.

48. The implant of claim 47 wherein the bearing surface is a polished surface.

49. The implant of claim 47 wherein the bearing surface has a surface roughness of less than about 2 micrometers.

50. The implant of claim 47 wherein the bearing surface has a sphericity of less than about 20 micrometers.

51. The implant of claim 47 wherein the carbon nanostructures have a diameter of less than about 185 nm.

52. The implant of claim 47 wherein the carbon nanostructures are nanotubes.

53. The implant of claim 47 wherein the carbon nanostructures are nanospheres.

54. The implant of claim 47 wherein the carbon nanostructures are nanofibers.

55. The implant of claim 47 wherein the nucleus is further constructed of PEEK.

56. A method of making an implant that provides for dynamic motion in the spine, the method comprising:
forming a body having a polymer matrix comprising less than about 15 percent by weight carbon nanofibers; and
forming a bearing surface on the implant, the bearing surface providing articulating motion between vertebral bodies.

57. The method of claim 56 wherein forming a body having carbon nanofibers comprises injection molding the body.

58. The method of claim 56 wherein forming a body having carbon nanofibers comprises forming the body from a carbon fiber reinforced PEEK.

59. The method of claim 56 wherein forming a bearing surface on the implant comprises polishing the bearing surface.
